(19) United States
(12) Patent Application Publication
(10) Pub. No.: US 2025/0152792 A1
(43) Pub. Date: May 15, 2025

(54) DIALYSIS SYSTEM AND METHODS

(71) Applicant: OUTSET MEDICAL, INC., San Jose, CA (US)

(72) Inventors: Dean HU, San Leandro, CA (US); Michael KIM, San Jose, CA (US); Steven M. MILLER, Palo Alto, CA (US)

(21) Appl. No.: 18/728,723

(22) PCT Filed: Jan. 12, 2023
(86) PCT No.: PCT/US2023/060575

§ 371 Date: Jul. 12, 2024

Related U.S. Application Data
(60) Provisional application No. 63/298,916, filed on Jan. 12, 2022.

(51) Int. Cl. A61M 1/16

(52) U.S. Cl. CPC A61M 1/1672; A61M 2205/10; A61M 2205/7545; A61M 2209/084

(57) ABSTRACT

Dialysis systems and methods are described which can include a number of features. The dialysis systems described can be to provide dialysis therapy to a patient in the comfort of their own home. The dialysis system can be configured to prepare purified water from a tap water source in real-time that is used for creating a dialysate solution. The dialysis systems described also include features that make it easy for a patient to self-administer therapy.

### CROSS REFERENCE TO RELATED APPLICATIONS

This application claims the benefit of priority of U.S. Provisional Application No. 63/298,916, filed Jan. 12, 2022, herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This disclosure generally relates to dialysis systems. More specifically, this disclosure relates to systems and methods for creating dialysate in real-time during dialysis treatment.

### BACKGROUND

There are, at present, hundreds of thousands of patients in the United States with end-stage renal disease. Most of those require dialysis to survive. Many patients receive dialysis treatment at a dialysis center, which can place a demanding, restrictive and tiring schedule on a patient. Patients who receive in-center dialysis typically must travel to the center at least three times a week and sit in a chair for 3 to 4 hours each time while toxins and excess fluids are filtered from their blood. After the treatment, the patient must wait for the needle site to stop bleeding and blood pressure to return to normal, which requires even more time taken away from other, more fulfilling activities in their daily lives. Moreover, in-center patients must follow an uncompromising schedule as a typical center treats three to five shifts of patients in the course of a day. As a result, many people who dialyze three times a week complain of feeling exhausted for at least a few hours after a session.

Many dialysis systems on the market require significant input and attention from technicians prior to, during, and after the dialysis therapy. Before therapy, the technicians are often required to manually install patient blood tubing sets onto the dialysis system, connect the tubing sets to the patient, and to the dialyzer, and manually prime the tubing sets to remove air from the tubing set before therapy. During therapy, the technicians are typically required to monitor venous pressure and fluid levels, and administer boluses of saline and/or heparin to the patient. After therapy, the technicians are often required to return blood in the tubing set to the patient and drain the dialysis system. The inefficiencies of most dialysis systems and the need for significant technician involvement in the process make it even more difficult for patients to receive dialysis therapy away from large treatment centers.

Given the demanding nature of in-center dialysis, many patients have turned to home dialysis as an option. Home dialysis provides the patient with scheduling flexibility as it permits the patient to choose treatment times to fit other activities, such as going to work or caring for a family member. Unfortunately, current dialysis systems are generally unsuitable for use in a patient's home. One reason for this is that current systems are too large and bulky to fit within a typical home. Current dialysis systems are also energy-inefficient in that they use large amounts of energy to heat large amounts of water for proper use. Although some home dialysis systems are available, they generally are difficult to set up and use. As a result, most dialysis treatments for chronic patients are performed at dialysis centers.

Hemodialysis is also performed in the acute hospital setting, either for current dialysis patients who have been hospitalized, or for patients suffering from acute kidney injury. In these care settings, typically a hospital room, water of sufficient purity to create dialysate is not readily available. Therefore, hemodialysis machines in the acute setting rely on large quantities of pre-mixed dialysate, which are typically provided in large bags and are cumbersome for staff to handle. Alternatively, hemodialysis machines may be connected to a portable RO (reverse osmosis) machine, or other similar water purification device. This introduces another independent piece of equipment that must be managed, transported and disinfected.

Dialysis machines are used in a variety of settings, including hospital rooms, dedicated clinics and patient homes. In some settings, minimal mobility requirements are needed, such as in the home or the clinic setting. In other settings, such as hospital rooms, mobility could be very important. The machine may need to be transported across long distances, hallways, or even exterior surfaces going from one building to another. Additionally, within a hospital room, space is at a premium, and high maneuverability e.g., ability to spin about its own axis, is desirable. However, mobility solutions that are optimized for one setting may not work well in other settings for size, footprint or cost reasons. Therefore, there is a need for a modular approach where a single dialysis machine could have an option of mobility solutions, and preferably where the installation of that modular mobility solution is minimally burdensome.

Preconfigured dialysis machines are those which have onboard water purification hardware, such as a reverse osmosis system. These systems often have a number of water filters, such as sediment, carbon and ultrafilters that purify the water that is later used to create dialysate. The quality of the incoming water has a significant impact on the life of many of these filters. Factors such as sediment content, chlorine/chloramine concentration, hardness, pH, alkalinity and temperature can shorten the lifespan of filters and/or impact the quality of the water after it is filtered. Due to highly varied nature of the incoming water, different options for treating the water would be desired. It could be conceivable to produce single a water treatment system that could handle a wide range of input variables, although doing so may be prohibitive from a size, weight, cost or maintenance standpoint. Therefore, there is a need for a modular approach for water prefiltration, and preferably one where maintenance such as changing filters is minimally burdensome.

Mobility for dialysis machines has been historically provided with casters. Typically these casters can be locked to brake the machine and prevent motion when it is not desired. If all four casters are allowed to rotate about their axis, the machine has high maneuverability in tight spaces. However, when transporting across long distances, the machine may have a tendency to rotate about its axis and be difficult to control. The need to allow the casters to swing through its arc also necessitates the need to create volume voids, or wheel wells, which limits the ability to minimize the size of the machine. Fixing at least one of the casters to not rotate can remedy the control issue, at the cost of close quarters maneuverability. Some casters have a ‘directional lock’ feature which allows them to be switched from freely rotating to fixed.

Water treatment carts have been known in the art, to provide portable water purification for dialysis machines. These tend to be very bulky and difficult to maneuver. The dialysis machines that they serve typically do not have their own onboard water filtration, so the water treatment carts typically perform all water filtration functions. For preconfigured dialysis machines that require a water connection, only supplemental water treatment is necessary, which may enable smaller form factors.

### SUMMARY

A dialysis system is provided comprising a dialysis console configured to provide dialysis therapy to a patient, and a modular platform configured to interface with the dialysis console, the modular platform including a fluid and/or power connection to the dialysis console, the modular platform further being configured to receive one or more modular components configured to add filtration and/or fluidic capabilities to the dialysis console.

In some embodiments, the dialysis console is configured to mount atop the modular platform.

In some examples, the modular platform further comprises a plurality of wheels.

In one embodiment, the modular platform includes one or more slots configured to receive the one or more modular components.

In some examples, the modular platform includes an electrical connection configured to mate with a corresponding electrical connection of the one or more modular components.

In some embodiments, the electrical connection is internal to the wheeled platform.

In one example, the electrical connection is on an exterior of the modular platform or dialysis console.

In some examples, the one or more modular components are configured to be electrically and fluidly coupled within the one or more slots by inserting the one or more modular components into the slots and rotating the one or more modular components by a preset angle. In one embodiment, the preset angle comprises up to 90 degrees. In other embodiments, the preset angle comprises up to 45 degrees. In some examples, the preset angle comprises 1-360 degrees.

In some embodiments, the one or more modular components include an exterior handle.

In some examples, the one or more modular components include a first filtration component and a second filtration component.

In one embodiment, the first filtration component comprises a filter and the second filtration component comprises a pump.

In another embodiment, the first filtration component comprises a first filter and the second filtration component comprises a second filter. In some examples, the filter, first filter, or second filter comprises a sediment filter, a carbon filter, a reverse osmosis filter, or an ultrafilter.

In some examples, the one or more modular components include a manifold configured to direct incoming water into a plurality of streams.

In some embodiments, the modular platform comprises a fluid inlet line configured to be coupled to a source of tap water and a fluid outlet line configured to be coupled to a fluid inlet line of the dialysis console.

In one embodiment, the dialysis console further comprises a fluid outlet line configured to be coupled to a drain.

In some examples, the dialysis console further comprises a fluid outlet line configured to be coupled to a drain-in line of the modular platform.

A method is provided, comprising receiving a flow of fluid from a fluid source into a modular platform that is separate from but fluidly coupled to a dialysis system, purifying the fluid with filters in the modular platform, and pumping the fluid from the modular platform to the dialysis system with a pump in the modular platform.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows one embodiment of a dialysis system.

FIG. 2 illustrates one embodiment of a water purification system of the dialysis system.

FIG. 3 illustrates one embodiment of a dialysis delivery system of the dialysis system.

FIGS. 4A-4E illustrate one embodiment of a dialysis system with a wheeled platform configured to house or accept modular dialysis system components.

FIG. 5A is another embodiment of a wheeled platform and modular dialysis system components.

FIG. 5B is one embodiment of a dialysis system configured to accept modular dialysis system components.

FIGS. 6A-6B illustrate another embodiment of a modular dialysis system.

FIG. 7 is one example of a fluid circuit enabled within a modular dialysis system.

FIG. 8 is a power schematic of a dialysis system with one or more modular components or a modular platform.

FIGS. 9A-9C illustrate various fluidic connections to a dialysis system.

FIGS. 10A-10B illustrate traditional filtration cartridges.

FIGS. 11A-11C illustrate a novel filtration cartridge.

FIG. 12 shows one specific embodiment of a novel filtration cartridge.

FIG. 13 is a system schematic of a dialysis system with five modular filtration components.

### DETAILED DESCRIPTION

This disclosure describes systems, devices, and methods related to dialysis therapy, including a dialysis system that is simple to use and includes automated features that eliminate or reduce the need for technician involvement during dialysis therapy. In some embodiments, the dialysis system can be a home dialysis system. Embodiments of the dialysis system can include various features that automate and improve the performance, efficiency, and safety of dialysis therapy.

In some embodiments, a dialysis system is described that can provide acute and chronic dialysis therapy to users. The system can include a water purification system configured to prepare water for use in dialysis therapy in real-time using available water sources, and a dialysis delivery system configured to prepare the dialysate for dialysis therapy. The dialysis system can include a disposable cartridge and tubing set for connecting to the user during dialysis therapy to retrieve and deliver blood from the user.

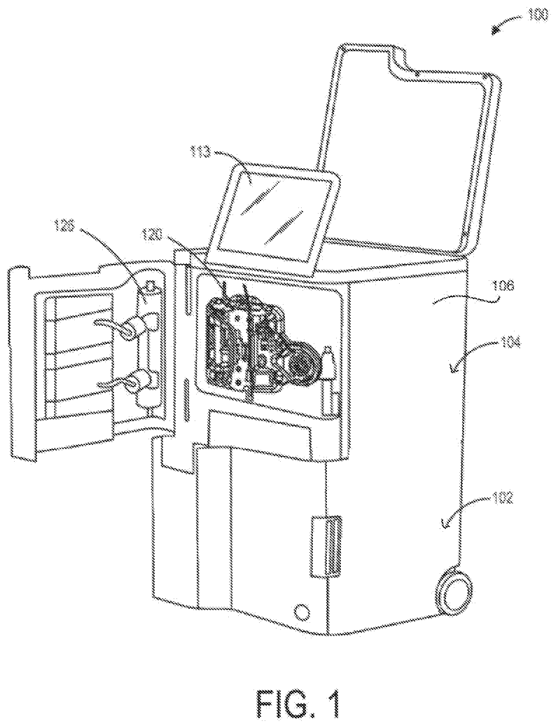

FIG. 1 illustrates one embodiment of a dialysis system 100 configured to provide dialysis treatment to a user in either a clinical or non-clinical setting, such as the user's home. The dialysis system 100 can comprise a water purification system 102 and a dialysis delivery system 104 disposed within a housing 106. The water purification system 102 can be configured to purify a water source in real-time for dialysis therapy. For example, the water purification system can be connected to a residential water source (e.g., tap water) and prepare purified water in real-time. The pasteurized water can then be used for dialysis therapy (e.g., with the dialysis delivery system) without the need to heat and cool large batched quantities of water typically associated with water purification methodologies.

Dialysis system 100 can also include a cartridge 120 which can be removably coupled to the housing 106 of the system. The cartridge can include a patient tubing set attached to an organizer. The cartridge and tubing set, which can be sterile, disposable, one-time use components, are configured to connect to the dialysis system prior to therapy. This connection correctly aligns corresponding components between the cartridge, tubing set, and dialysis system prior to dialysis therapy. For example, the tubing set is automatically associated with one or more pumps (e.g., peristaltic pumps), clamps and sensors for drawing and pumping the user's blood through the tubing set when the cartridge is coupled to the dialysis system. The tubing set can also be associated with a saline source of the dialysis system for automated priming and air removal prior to therapy. In some embodiments, the cartridge and tubing set can be connected to a dialyzer 126 of the dialysis system. In other embodiments, the cartridge and tubing set can include a built-in dialyzer that is pre-attached to the tubing set. A user or patient can interact with the dialysis system via a user interface 113 including a display.

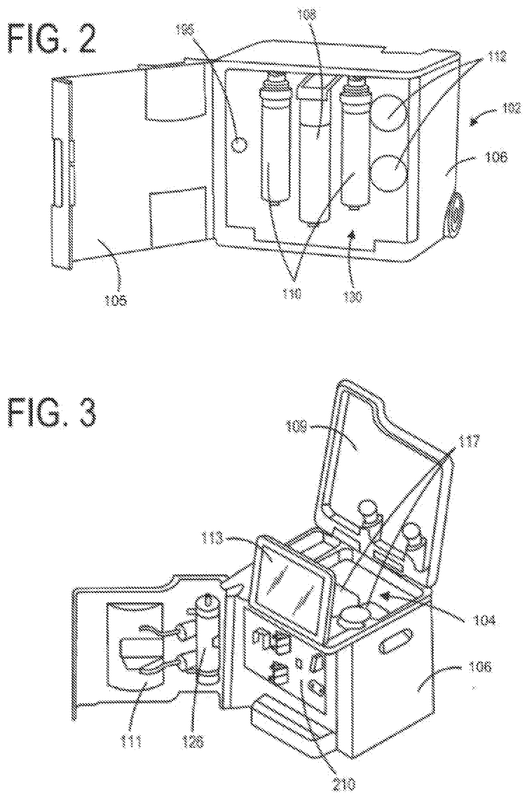

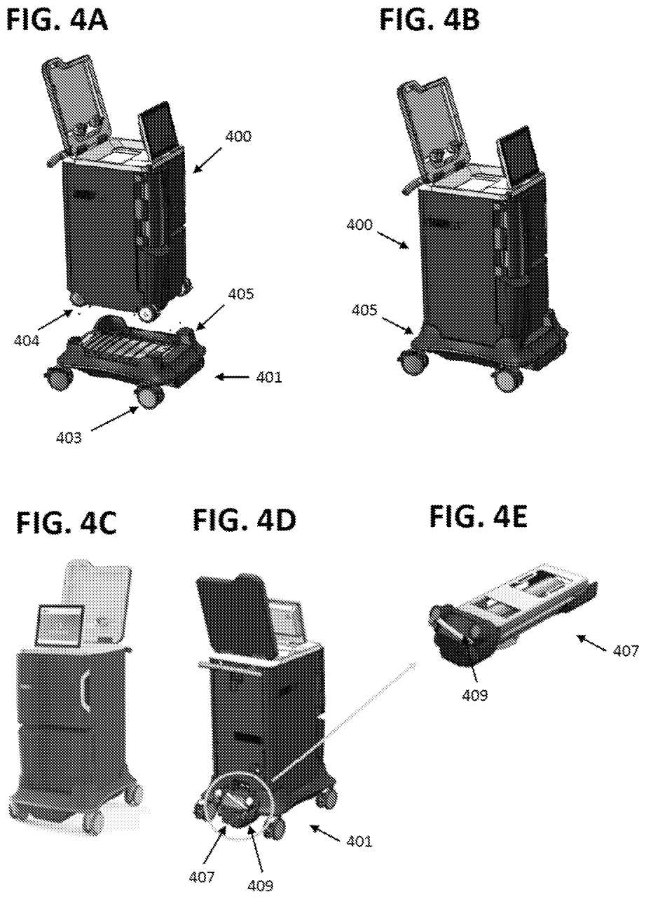

FIGS. 2-3 illustrate the water purification system 102 and the dialysis delivery system 104, respectively, of one embodiment of the dialysis system 100. The two systems are illustrated and described separately for ease of explanation, but it should be understood that both systems can be included in a single housing 106 of the dialysis system. FIG. 2 illustrates one embodiment of the water purification system 102 contained within housing 106 that can include a front door 105 (shown in the open position). The front door 105 can provide access to features associated with the water purification system such as one or more filters, including sediment filter(s) 108, carbon filter(s) 110, and reverse osmosis (RO) filter(s) 112. The filters can be configured to assist in purifying water from a water source (such as tap water) in fluid communication with the water purification system 102. The water purification system can further include heating and cooling elements, including heat exchangers, configured to pasteurize and control fluid temperatures in the system, as will be described in more detail below. The system can optionally include a chlorine sample port 195 to provide samples of the fluid for measuring chlorine content.

In FIG. 3, the dialysis delivery system 104 contained within housing 106 can include an upper lid 109 and front door 111, both shown in the open position. The upper lid 109 can open to allow access to various features of the dialysis system, such as user interface 113 (e.g., a computing device including an electronic controller and a display such as a touch screen) and dialysate containers 117. Front door 111 can open and close to allow access to front panel 210, which can include a variety of features configured to interact with cartridge 120 and its associated tubing set, including alignment and attachment features configured to couple the cartridge 120 to the dialysis system 100. Dialyzer 126 can be mounted in front door 111 or on the front panel, and can include lines or ports connecting the dialyzer to the prepared dialysate, dialysate concentrate, liquid concentrate, etc., as well as to the tubing set of the cartridge. In one implementation, described in more detail below, the dialysate machine and/or dialyzer can include lines or ports configured to connect to a disposable reservoir that includes a plurality of powdered and/or liquid compartments for dialysate preparation and delivery.

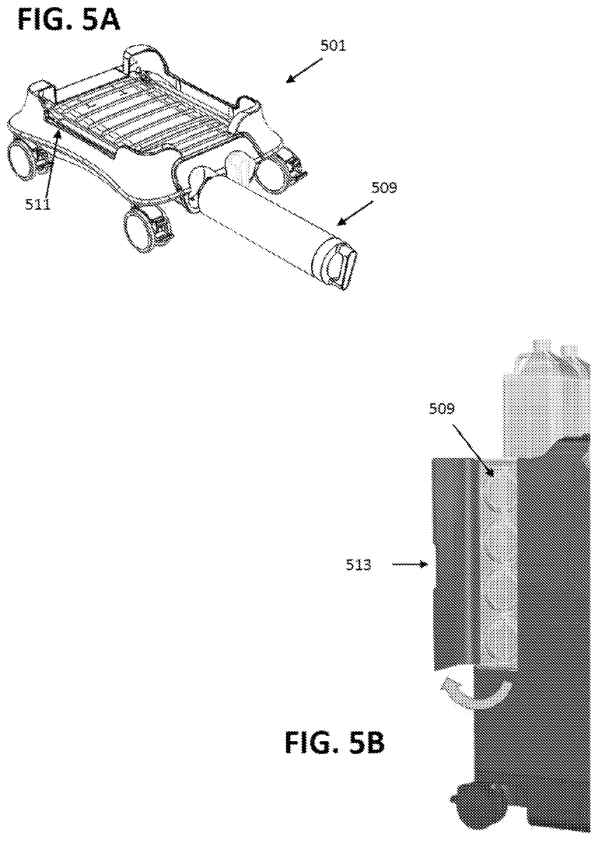

In one embodiment, referring to FIGS. 4A-4E, a wheeled platform 401 is provided upon which a preconfigured dialysis system 400 (such as the dialysis system 100 of FIG. 1) may be mounted on. FIG. 4A shows the wheeled platform 401 separate from the dialysis system 400, and FIGS. 4B-4C show the dialysis system mounted on the wheeled platform.

As shown, the wheeled platform 401 can include a plurality of wheels 403 configured to provide additional maneuverability and/or mobility to the dialysis system when the wheeled platform is engaged with the system. It should be understood that while the dialysis system itself may have two or more wheels 404 for portability, these wheels typically involve shortcomings as described above that really limit the maneuverability of the system. The entire dialysis system, including the built-in wheels 404, are configured to fit within, connect to, or mate with the wheeled platform. In some embodiments, the dialysis system has wheels of its own, which do not need to be removed in order to mount it to the wheeled platform. In some embodiments, the wheels of the wheeled platform are larger and more robust than the wheels of the dialysis machine. For example, the wheel diameter of the dialysis machine may be 4 inches or less, and the wheel diameter of the wheeled platform may be 5 inches or more. The increase in size will allow the wheeled platform to more easily traverse obstacles such as thresholds or uneven surfaces. The wheeled platform may also have a wider wheelbase than the dialysis machine, in order to provide improved stability.

The dialysis machine can be securely and rigidly fastened to the platform by either its wheels, some other mounting feature designed for this purpose. In some embodiments, the wheeled platform 401 can be connected to the dialysis machine with a threaded connection, a quick release connection, magnets, or the like. The wheeled platform 401 in some embodiments has shrouds 405 which can be configured to attach to, mate to, and or connect with the wheels 404 of the dialysis system. In some embodiments, the shrouds 405 may effectively hide the wheels 404 of the dialysis machine visually, to promote a seamless look. Additionally, any service activities that would need to be performed on the dialysis machine that involve removing panels from the dialysis machine can be performed while the machine is mounted to the wheeled platform 401. Therefore, the wheeled platform can be specifically contoured to the dialysis system so as to not prevent those panels from being removed. Alternatively, the wheeled platform itself can have removable panels that allow for removal of all panels of the dialysis system.

As will be described in more detail below, referring to FIGS. 4D-4E, the wheeled platform 401 may further be configured to house or accept modular dialysis system components that expand or enrich the functionality of the dialysis system when mounted to the wheeled platform. In FIGS. 4D-4E, the wheeled platform includes an enhancement module 407 configured to expand and enrich the functionality of the dialysis system. In some embodiments, the enhancement module is removable/insertable from the wheeled platform 401. When the enhancement module is removed from the wheeled platform, the individual components within the enhancement module can be accessed, serviced, or replaced. When the enhancement module is inserted into the wheeled platform 401, the additional functionality provided by the enhancement module can be automatically detected and activated by the dialysis system.

In some embodiments, the enhancement module 407 can comprise water treatment equipment (e.g., water filters, ultrafilters, carbon filters, etc.), pumping equipment (e.g., additional pumps), dialysis/dialysate equipment (e.g., dialyzers, additional dialyzers, drip chambers, air removal systems, priming fluid, etc.), waste management equipment (e.g., waste containers for used dialysate or blood waste), filtering equipment, etc., or some combination thereof, and can include both fluidic and electrical connections to both the dialysis machine and wall (power and water). The electrical connections between the wheeled platform and the dialysis system may further comprise one or both of data transfer and/or power transfer. Additionally, the enhancement module 407 can include a user interface 409 which can optionally include a display. In some embodiments, the display can provide information to the user regarding the functionality of the enhancement module components.

In one specific embodiment, as shown in FIG. 4E, the enhancement module 407 can include a water pre-treatment module that can include filters (e.g., sediment and chlorine variants), increased filtering and/or water treatment capacity over filters found in traditional dialysis systems, an optional pressure booster pump, and the capacity to integrate a pH balance system, among other features.

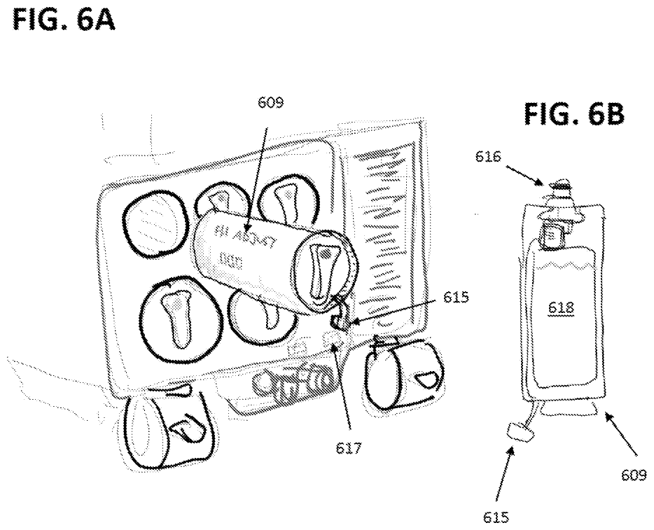

In other embodiments, the wheeled platform does not include a removable enhancement module as described above, but is instead configured to directly receive specific modular components for enhancing the functionality of the dialysis system. Referring to FIG. 5A, the wheeled platform 501 can be configured to receive removable modular components 509. Furthermore, additional space 511 within the wheeled platform 501 may be dedicated to other modular dialysis components (not shown) which are not removable. For example, the wheeled platform can include permanent components such as batteries, pumps, sensors, or filter cartridges. For example, a fixed booster pump may be integrated into the wheeled platform, along with valves and sensors to monitor the performance of the water treatment functionality. As another example, pressure sensors can be integrated into the wheeled platform and be configured to monitor a pressure drop across filters within the base. Additionally, removable filters may be integrated into the base, and a visual indicator on the exterior of the wheeled platform or on the console of the dialysis system may provide indication of remaining filter life to the user. Alternatively, the wheeled platform may comprise or include one or more microprocessors capable of communicating with the dialysis machine that it is connected to, either wirelessly or via a wired connection between the wheeled platform and the dialysis system. The firmware on the dialysis machine can be configured to interprete the signals sent by the microprocessor(s) in the wheeled platform and display a status on the GUI of the dialysis machine or otherwise raise an alarm. The microprocessor may also communicate wirelessly with a remote server or other device remotely to provide status of performance of the water filtration.

In some embodiments, the modular components are optional. In some cases where the water treatment functionality built-in to the dialysis machine is adequate, the machine may still benefit from improved mobility afforded by the separate wheeled platform. In these cases, the volume within the wheeled platform may be empty, and can instead be used for storage. For example, the storage area within the wheeled platform could be accessed by a sliding drawer or hinged door, for example.

In another embodiment, referring to FIG. 5B, the dialysis machine itself can be configured to accept the modular dialysis system components 509, which can be accessed via drawer or door 513 on the dialysis machine.

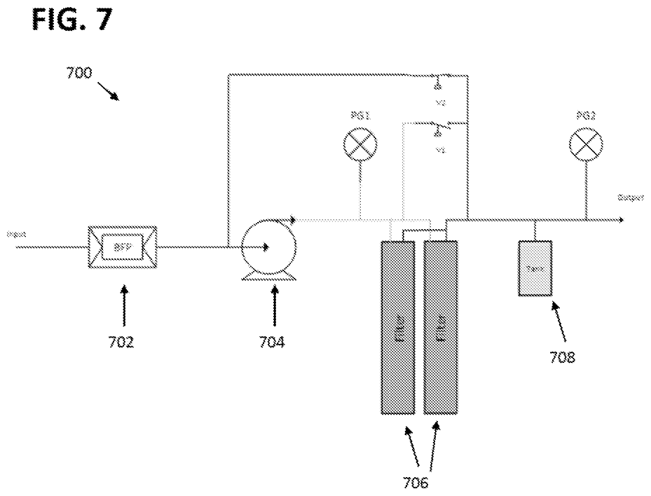

Referring to FIGS. 6A-6B, and as mentioned above, in some embodiments the modular dialysis components 609 can include an electrical connection 615 to the wheeled platform (or directly to the dialysis machine). In one embodiment, as shown in FIG. 6A, the electrical connection 615 can be on a front portion of the modular component and can be configured to plug into or be electrically connected to a corresponding electrical connection 617 on the wheeled platform (or the dialysis machine). In other embodiments, the electrical connection can be made automatically when the modular component is inserted into the wheeled platform or dialysis machine. For example, an electrical contact or connection on a back of the modular component can be configured to electrically mate with a corresponding electrical contact or connection inside the wheeled platform or dialysis machine.

Referring to FIG. 6B, the modular component can also include one or more fluidics connections 616. The fluidics connection(s) 616 can provide fluid communication between a modular component such as a filter or fluid reservoir 618 and a source of fluid (e.g., tap water) and/or the dialysis machine, as will be described in more detail below. In this example, when the modular component is inserted into the wheeled platform or dialysis machine, the fluidics connection can automatically be made between the modular component, the wheeled platform, the dialysis machine, and/or a source of fluid.

The modules could be simple flow through filters, or they could have electronic components (pumps, valves, sensors). For example, one module could be a chemical reservoir with a low flow feed pump that injects fluid into the water to treat it. By plugging the connector in, it could also help to identify what type of module is being inserted.

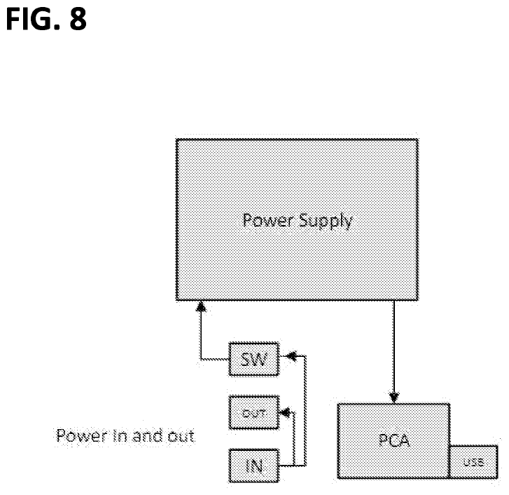

FIG. 7 is a schematic drawing showing one embodiment of a fluid circuit 700 within a dialysis system that includes modular components as described herein and above. In one example, dialysis machine can be mounted on the wheeled platform and be configured to take incoming water and produce dialysate. The water can be routed through the fluidic modular components within the wheeled platform. Under operating conditions, the incoming water passes through the fluidic modular components which can include one or more filters, pumps, valves and/or sensors. For example, a booster pump may be provided in on or more of the modular components to increase the pressure of the incoming water, which may be helpful in producing sufficient flow across filtration media of the fluidic modular components and/or the filtration media on or within the dialysis machine itself.

FIG. 7 is a basic flow diagram of the fluidic modular components which comprise two filters. It should be understood that other embodiments can include any number of filters, including one filter or more than two filters. Incoming water can pass through a backflow preventor 702. A booster pump 704 can included in one or more of the modular components and can be configured to boost pressure to the two filters 706. The two filters may be arranged in series or in parallel, depending on the need. Preferably, the filters may be switched between parallel and series configuration by, for example, a manual valve.

The pressure drop across the filters 706 can be measured by pressure gauges PG1 and PG2. In some embodiments, the pressure gauges can be integrated into the modular components. In other embodiments, the pressure gauges can be permanently located within the dialysis system or the wheeled platform. The pressure measurements can be used by microprocessors or controllers of the system to determine the pressure drop across the filters. If the pressure drop is excessive, a proportional valve VI may be opened, which bypasses the filters within the fluidic module, and sends the incoming water directly to the dialysis machine, which has its own filters, VI can be a proportional valve, where the degree to which it is opened (and thus the amount of water sent to the filters vs. sent directly to the dialysis machine) may be varied depending on the pressure drop in the filters. In one example, the pump 704 may be set to a pressure control mode, where a target pressure at PG2 is desired. If no water is demanded from the fluidic module by the dialysis machine, for example, if the dialysis machine is powered off, then PG2 will reach a nominal pressure and the pump will shut off. Valve V2 is another bypass valve, which is configured to be normally open, such that when it is not powered, it is opened. In this manner, if the fluidic module is not supplied power, the incoming water may directly flow to the dialysis machine and not be obstructed by the powered down pump. A check valve may be included which allows the system to hold pressure in a no flow condition. Optionally, backflow prevention such as a series of check valves or reduced pressure zone assembly may be provided upstream of all functions. A tank 708 can be used to control dP/dT and can also handle pressure spikes within the system.

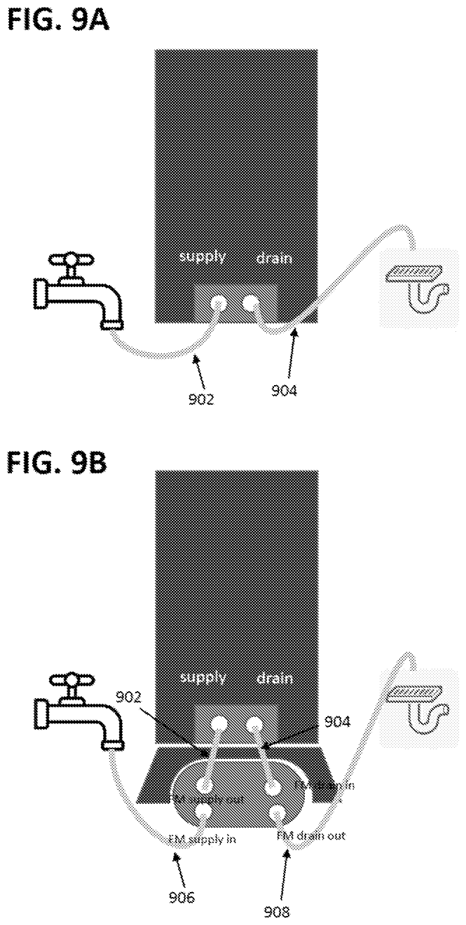

The dialysis machine and the fluidic module of the wheeled platform to which the dialysis machine is mounted can both include an electrical connection. In one embodiment, referring to FIG. 8, a single power cord to the wall can be configured to supply power to both the dialysis machine and the fluidic module/wheeled platform. Even if not connected to the wheeled platform and fluidic module, the dialysis machine has its own power connection and power supply. Preferably, a power cord from the mains will connect to the power supply IN of the fluidic module, and supply power to it (via a switch SW). From there, the fluidic module or the power cord can also include a power out (OUT) with direct connection to the power of the dialysis system, from which a second power cord would connect from the fluidic module into the power connector of the dialysis machine. The power supply of the fluidic module would supply power to a printed circuit assembly (PCA), which would then distribute power to the various components. The PCA can reside within the wheeled platform described above. The PCA may also have USB connections which may be leveraged for expandability, for example a wireless communication module, or a memory device capable of storing logged pressure or other data.

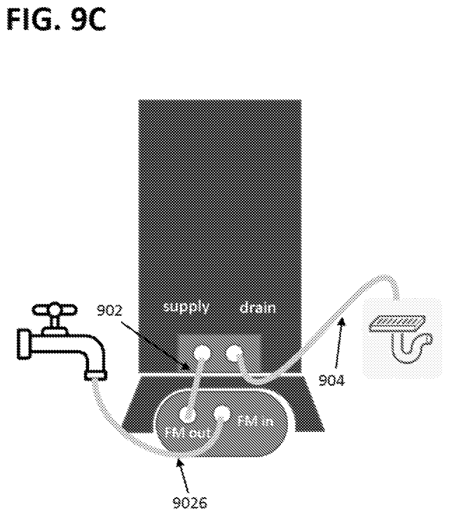

The dialysis system and wheeled platform can further include various fluidic connections to an outside fluid source (such as a tap water source). Referring to FIG. 9A, the dialysis machine can include a fluid supply line 902 to provide fluid/tap water into the system and a drain line 904 to dispose of spent fluid/dialysate. These connections are made directly to the dialysis machine. FIG. 9B shows an embodiment when the dialysis machine is connected to or mated with the wheeled platform described above. In this example, the wheeled platform includes a fluid supply in line 906, which has a corresponding fluid supply out line 908 which connects directly to the fluid supply line 902 of the dialysis machine. Similarly, the drain line 904 of the dialysis machine is fluidly coupled to a drain line in of the wheeled platform, which is then fluidly connected to a corresponding drain out line of the wheeled platform for disposing of spent fluid/dialysate.

In another example, referring to FIG. 9C, the drain out line 904 of the dialysis system does not pass through the wheeled platform, but instead goes straight to drain. In this configuration, water enters the wheeled platform from the water source through fluid supply in line 906, is fluidly coupled to the supply line 902 of the dialysis system, and goes directly to drain from the drain line 904 of the dialysis system.

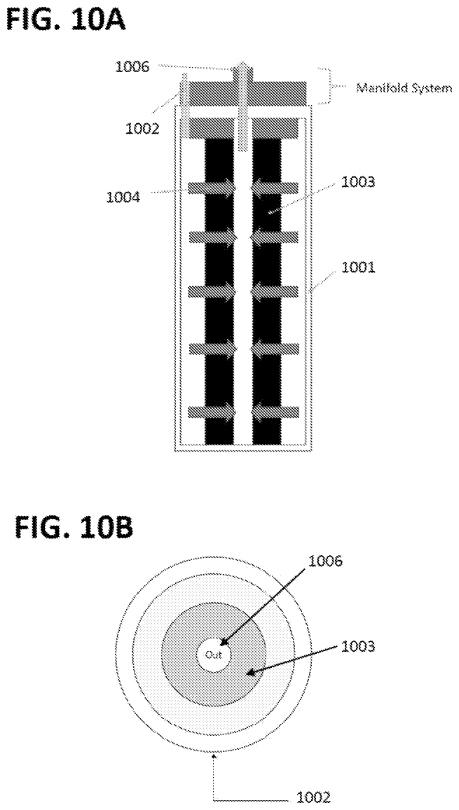

This disclosure also provides novel filtration cartridges that can be used in any of the systems and embodiments described above, including within the wheeled platform or as modular filtration components. FIGS. 10A-10B show one example of a traditional filtration cartridge 100 including a filter housing 1001 and a filter media 1003. In this example, the filtration cartridge can include an inlet 1002 for incoming water which then passes radially through one or more filtration media in the cartridge indicated by arrows 1004. The filtered water then passes back out centrally through an outlet 1006 in the cartridge. As shown in FIG. 10B, multiple filtration media can be separated diametrically to accommodate different filter media types.

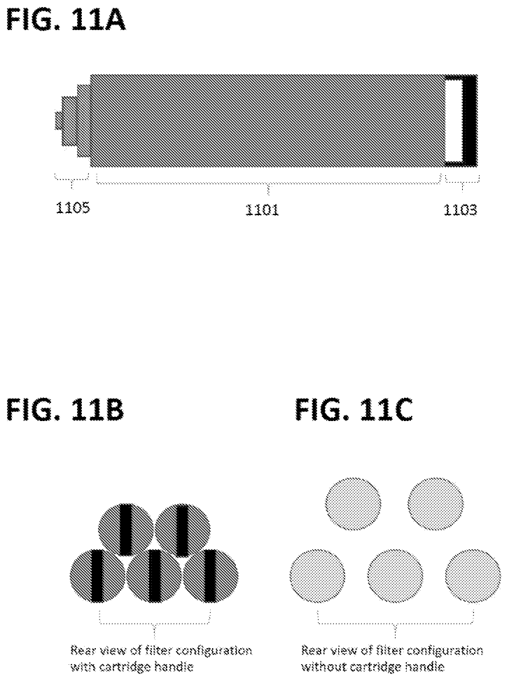

FIGS. 11A-11C illustrate one embodiment of a modular filter component that can be used in conjunction with the wheeled platform and/or dialysis systems described herein. As mentioned above, the modular components can provide additional functionality to the dialysis system/wheeled platform, including filtering capabilities. Additionally, the modular components can include an electrical connection. The novel water filtration system described in this disclosure centers around a common housing design. The water filter uses a common large size enclosure 1101 but adds a handle 1103 at the end of the filter (as shown FIGS. 11A-11B). Having the handle on the rear of the filter cartridge eliminates the need to have spacing between the filters (for the physical space required for hands) to service the filters. Therefore, the handle on the filters allows the positioning of multiple filters to be in very close proximity with each other. This reduces space claimed within the system and minimizes the fluid connection line lengths between each filter. FIG. 11B shows the spacing that is enabled by adding the handle to the cartridge, while FIG. 11C shows the spacing that would be required without the handle. This allows for packing more cartridges into a smaller space (such as in the wheeled platform) than would otherwise be possible.

The front of the modular filter component in FIG. 11A can include a filter head 1105 configured to allow up to a three-stream inlet/outlet for fluid flow by creating concentric and semi-concentric openings. Using a multi-layered filter head (shown in FIG. 11A) with decreasing diameters leading up to the tip of the filter head, each layer can be used to create a new flow path with corresponding sealing mechanisms (e.g., radial O-rings) between each layer.

A manifold connection system can be utilized to connect the front of the filter cartridges to the overarching fluidic system. The entryway to the manifold system can contain a steep incline step at the connection site. Several of these incline steps coupled with the handle at the other end of the filter allows for a smooth 90-degree locking and unlocking feature of the cartridge filter (inside the manifold).

The housing size of the modular filter component can accommodate various filter media/core types to add flexibility within the water filtration system. A balancing of equations, with the life and size of various filters media cores, is required to find a common volume which encompasses all water specification requirements. In some embodiments, a single large filter housing can be used to separate and house two different types of media cores by utilizing the large space and the housing construction.

In one example, the multi-media core can be arranged in the common concentric separation shown in FIG. 11C. This type of construction uses the actual filter media core to wrap around each other. The multi-media core can be sustained by the filter media/core. It should be noted that these tightly wrapped media cores can only be applied in normal filter media types (e.g., carbon, sediment, RO).

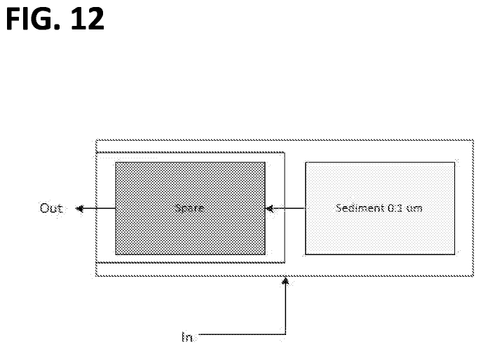

In another embodiment, a second type of multi-media core can be provided within the housing which comprises a two-section media/core that utilizes the housing of the filter to separate the cores. This configuration is shown in FIG. 12. By building the construction of the filter housing in a way that creates a flow path from the inlet to bypass a filter core to create a flow path, a second type of multi-media core can be contained within a single housing. It should be noted that the arrows don't dictate the physical ports of the input/outputs for the flow. Furthermore, all filters notated in this embodiment and in this disclosure can have the inlet and outlet (for the flow) concentrated around the filter head shown in FIG. 11A.

The multi-media core within the filter housing can accommodate various modules required for the water filtration system. For the purposes of the illustrations presented in FIG. 12, the “Spare” portion of the filter can represent any type of module, part, system, or electronics that can be inserted in the allocated space within the filter housing partition. Either one of these two multi-media core options can be used to create an optimized multi-media filter system optimized for manufacturing and tailored to a specific need.

These large filter housings can accommodate various types of filter cores (both single and multi-media cores) and internal components. By controlling the water flow path using the filter housing, other types of modules can be incorporated within the housing core mimicking tubing within a flow path. Additional modules can be put into a section of the filter housing to accommodate various water filtration system needs. These modules can include (but not limited to) a fluid pump to control flow rate without the water filtration system, injection system to control water pH, and even electronics. Referring to FIG. 12, it should be understood that these modules such as pumps, electronics, or injection systems could be positioned in the “spare” slot within the modular component, with a filtering component such as a sediment filter positioned in the other location within the housing. Other types of filtering media can be used in the “sediment” position as well.

To accommodate any given electronics within the filtration system, one of two different types of connections may be used. The first option would be a blind connection with the proper structural lead-in, which can be accommodated because the forced orientation of the handle which enables a default 90-degree turn. The predetermined orientation of the filter cartridge along with 90-degree turn/locking mechanism allows for enough control to create a blind connection. A second option would be to have an end connector on the handle area of the filter cartridge. A female or male connection point can be put onto the cartridge (rear) cap area to connect the electronic connections. One example of the end connectors is shown in the embodiment of FIGS. 6A-6B.

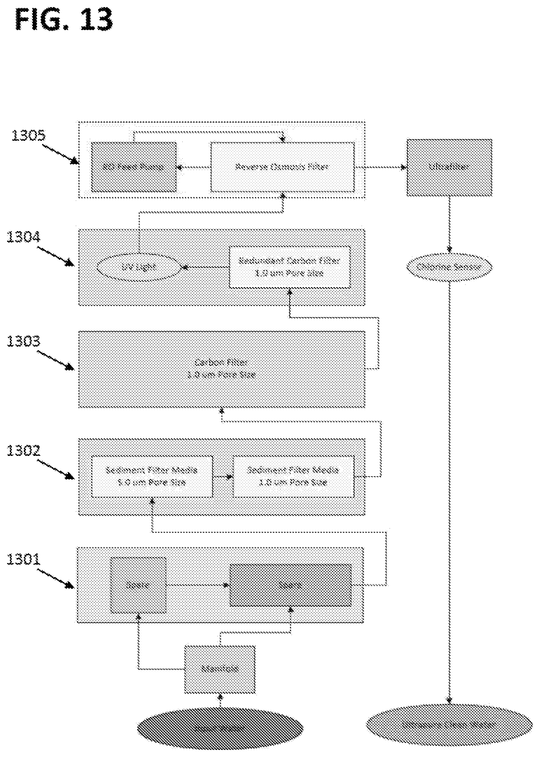

FIG. 13 illustrates one example of a multi-cartridge filtration water system that includes the concepts described above. This system can bring about various configurations and flexibility within a water filtration system. By putting multiple filters in line, a combination of single media core and multi-media cores can be arranged to bring about the most optimized water filtration system. With the ability of the wheeled platform and/or dialysis system described above to receive a plurality of modular components, numerous configurations and possibilities are enabled by the present disclosure.

For example, referring to FIG. 13, this embodiment includes a water filtration system with a total of five modular components or cartridges. These components can be inserted into a wheeled platform which is then coupled to a dialysis system. Alternatively, as illustrated in FIG. 5B, the modular components can be inserted directly into corresponding slots directly on the dialysis system. In FIG. 13, first modular component 1301 can include first and second spare slots. While the exact purpose of these slots aren't defined in this example, it should be understood that these spare slots can provide any desired purpose, including filtration. It should be noted that the manifold can provide dual input streams of water directly to each of the spare slots. Additionally, the spare slots can be arranged in series, so the output of the first spare slot goes into the second spare slot, which then provides an output to second modular component 1302. Second modular component 1302 can include first and second sediment filter media with varying pore sizes, such as 5.0 μm and 1.0 μm. These can be arranged in series, as shown. The output from this modular component can be provided to third modular component 1303, which can comprise a carbon filter. Next, the output can be provided to fourth modular component 1304, which can include, for example, a redundant carbon filter and a UV light arranged in series. Finally, the fifth modular component 1305 can include a reverse osmosis filter and a feed pump configured to draw fluid from the filter and feed it back into the reverse osmosis filter. The output from all these modular components can optionally be passed through an ultrafilter and then a chlorine sensor. In some embodiments, the ultrafilter/sensor can be disposed in the modular components, and in other embodiments they can be disposed in the wheeled platform or dialysis system. The result of this filtration system is ultrapure clean water suitable for use by the dialysis system for dialysis therapy.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. A dialysis system comprising:
a dialysis console configured to provide dialysis therapy to a patient;
a modular platform configured to interface with the dialysis console, the modular platform including a fluid and/or power connection to the dialysis console, the modular platform further being configured to receive one or more modular components configured to add filtration and/or fluidic capabilities to the dialysis console.

2. The dialysis system of claim 1, wherein the dialysis console is configured to mount atop the modular platform.

3. The dialysis system of claim 1, wherein the modular platform further comprises a plurality of wheels.

4. The dialysis system of claim 1, wherein the modular platform includes one or more slots configured to receive the one or more modular components.

5. The dialysis system of claim 1, wherein the modular platform includes an electrical connection configured to mate with a corresponding electrical connection of the one or more modular components.

6. The dialysis system of claim 1, wherein the electrical connection is internal to the wheeled platform.

7. The dialysis system of claim 1, wherein the electrical connection is on an exterior of the modular platform or dialysis console.

8. The dialysis system of claim 4, wherein the one or more modular components are configured to be electrically and fluidly coupled within the one or more slots by inserting the one or more modular components into the slots and rotating the one or more modular components by a preset angle.

9. The dialysis system of claim 8, wherein the preset angle comprises up to 90 degrees.

10. The dialysis system of claim 8, wherein the preset angle comprises up to 45 degrees.

11. The dialysis system of claim 8, wherein the preset angle comprises 1-360 degrees.

12. The dialysis system of claim 1, wherein the one or more modular components include an exterior handle.

13. The dialysis system of claim 1, wherein the one or more modular components include a first filtration component and a second filtration component.

14. The dialysis system of claim 13, wherein the first filtration component comprises a filter and the second filtration component comprises a pump.

15. The dialysis system of claim 13, wherein the first filtration component comprises a first filter and the second filtration component comprises a second filter.

16. The dialysis system of claim 14, wherein the filter, first filter, or second filter comprises a sediment filter, a carbon filter, a reverse osmosis filter, or an ultrafilter.

17. The dialysis system of claim 13, wherein the one or more modular components include a manifold configured to direct incoming water into a plurality of streams.

18. The dialysis system of claim 1, wherein the modular platform comprises a fluid inlet line configured to be coupled to a source of tap water and a fluid outlet line configured to be coupled to a fluid inlet line of the dialysis console.

19. The dialysis system of claim 18, wherein the dialysis console further comprises a fluid outlet line configured to be coupled to a drain.

20. (canceled)

21. A method, comprising:
receiving a flow of fluid from a fluid source into a modular platform that is separate from but fluidly coupled to a dialysis system;
purifying the fluid with filters in the modular platform; and
pumping the fluid from the modular platform to the dialysis system with a pump in the modular platform.
